(19) United States
(12) Patent Application Publication
(10) Pub. No.: US 2006/0264823 A1
(43) Pub. Date: Nov. 23, 2006

(54) Controlled failure balloon catheter assemblies

(76) Inventors: Kenneth R. Newcomb, Wilmington, DE (US); Jacob J. Maniyatte, Newark, DE (US)

Correspondence Address: GORE ENTERPRISE HOLDINGS, INC., 551 PAPER MILL ROAD, P. O. BOX 9206, NEWARK, DE, 19714-9206, US

(21) Appl. No.: 11/493,691

(22) Filed: Jul. 25, 2006

Related U.S. Application Data
(62) Division of application No. 11/037,747, filed on Jan. 18, 2005.

(51) Int. Cl. A61M 29/00

(52) U.S. Cl. 604/99.01; 604/118; 604/103.04

(57) ABSTRACT

Novel balloon catheter assemblies comprising controlled failure portion that fails at a pressure which will prevent undesired balloon rupture. The controlled failure portion can be located on or in a catheter shaft, a hub assembly, and/or a hub extension piece.

### RELATED APPLICATIONS

The present application is a divisional application of commonly owned and co-pending U.S. patent application Ser. No. 11/037,747, filed Jan. 18, 2005.

### FIELD OF THE INVENTION

The present invention relates to novel balloon catheter assemblies.

### BACKGROUND OF THE INVENTION

Balloon catheters are used for a variety of medical procedures. Their conventional use entails the insertion of the balloon catheter into a body conduit at a cannulation site and pushing the length of the catheter progressively into the body conduit until the balloon located at the distal end of the balloon catheter reaches the desired site. The balloon is then inflated at that site in order to implement the desired therapy. The body conduit is most often a blood vessel and more particularly an artery, although balloons are used within a variety of other body conduits such as, for example, bile ducts. The inflation of the balloon may be used for various therapeutical reasons such as causing temporary occlusion of the body conduit, for the delivery of a medicant to the specific site of inflation, to disrupt plaque or thrombus or to deliver a device to a desired site within the body conduit. Devices most commonly delivered with a catheter balloon include vascular stents, vascular stents in combination with vascular grafts (stent-grafts), and intraluminal vascular grafts, all of which may be circumferentially distended by inflation of the balloon until the device is implanted in firm contact with the wall of the body conduit. Typically, such balloon catheters will be advanced through the lumen of a previously placed guide catheter. A relatively short portion of the distal portion of the balloon catheter is advanced beyond the distal end of the guide catheter, while a relatively longer portion of the catheter is located within the guide catheter.

FIG. 1 shows, in schematic view, a representation of a typical balloon catheter device. The balloon catheter 1 includes hub assembly 2 at the proximal end of the catheter 1. Hub assembly 2 includes guide wire receiving port 5, which is in communication with a guide wire lumen which extends to the distal tip 8 of the catheter. Also included in hub assembly 2 is inflation port 4, which is in communication with an inflation lumen which extends from port 4 through the hub assembly 2, through the catheter shaft and into fluid communication with the interior of inflatable balloon 7.

Inflation fluid is introduced through inflation port 4, through the inflation lumen and into the interior of balloon 7 to inflate the balloon. To deflate the balloon, inflation fluid is simply removed through inflation port 4.

In use, catheter balloons (and particularly dilatation balloons) are known to occasionally rupture due to inflation to higher than design pressures. Sudden rupture and corresponding sudden release of inflation pressure can result in damage to the surrounding body conduit. Even if the balloon remains intact following rupture, the configuration of the damaged balloon may make withdrawal of the balloon from the body conduit quite difficult. These occasional ruptures can also result in fragmentation of the balloon and the necessity to retrieve the fragments. Due to displacement of the fragments distally as a result of fluid flow through the body conduit, retrieval is difficult at best and may require interventional surgery. It may not be possible to know with certainty that all pieces have been retrieved.

Thus, the art would benefit from a catheter assembly that provides a mechanism that will safely release inflation fluid either outside of the patient's body or within the guide catheter (when used) before a pressure is reached that would cause balloon rupture.

### SUMMARY OF THE INVENTION

Disclosed are balloon catheter assemblies comprising a catheter shaft having a proximal end and a distal end, an inflation lumen extending from the catheter shaft proximal end to the distal end, and being in fluid communication with the interior of an inflatable balloon located on the distal end of the catheter shaft. The inflation lumen being defined by a catheter wall portion, the catheter wall portion including at least one controlled failure portion preferably located sufficiently proximate to the inflatable balloon such that the at least one controlled failure portion will remain inside a guide catheter (when a guide catheter is used) or outside the patient's body when a procedure is performed. The catheter shaft can also comprise a guide wire receiving lumen extending for at least a portion of the length of the catheter shaft.

Also disclosed are hub assemblies for use with balloon catheters. The hub assembly comprising: a hub proximal end and a hub distal end; an inflation lumen extending from the hub proximal end to the hub distal end, the inflation lumen being defined by a hub wall portion; wherein the hub wall portion includes at least one controlled failure portion.

The hub assembly can further comprise a guide wire receiving lumen extending from the hub proximal end to the distal end. Moreover, the hub assembly can be joined to a catheter shaft having a proximal end and a distal end and an inflation lumen extending from the proximal end to a point distal thereto, wherein the catheter shaft proximal end is joined to the hub assembly, the catheter shaft inflation lumen being in fluid communication with the hub assembly inflation lumen, and wherein an inflatable balloon having an interior space is located on the catheter shaft distal end and the inflation lumen is in fluid communication with the interior space of the inflatable balloon.

Further disclosed are hub extension pieces having an inflation lumen defined by a wall portion, the wall portion comprising at least one controlled failure portion. The hub extension piece can be positioned proximate to a hub assembly such that the hub assembly inflation lumen is in fluid communication with the hub extension piece inflation lumen.

### BRIEF DESCRIPTION OF THE DRAWINGS

FIG. 1 is a schematic drawing of a prior art balloon catheter device.

FIG. 2 is a schematic drawing of a balloon catheter device and hub assembly according to the present invention.

FIG. 3 is a schematic cross-section of a portion of a catheter shaft that includes controlled failure portion according to the present invention;

FIG. 4A is a schematic cross-sectional view of a further controlled failure portion located on a catheter shaft wall according to the present invention;

FIG. 4B is a schematic cross-section of a further embodiment showing controlled failure portion contacting a wall portion of the catheter shaft;

FIG. 5 is a schematic cross-section of a hub assembly according to the present invention.

FIG. 6 is a schematic drawing of a hub assembly and hub extension device according to the present invention.

FIG. 7 shows in schematic cross-section a further hub assembly according to the present invention.

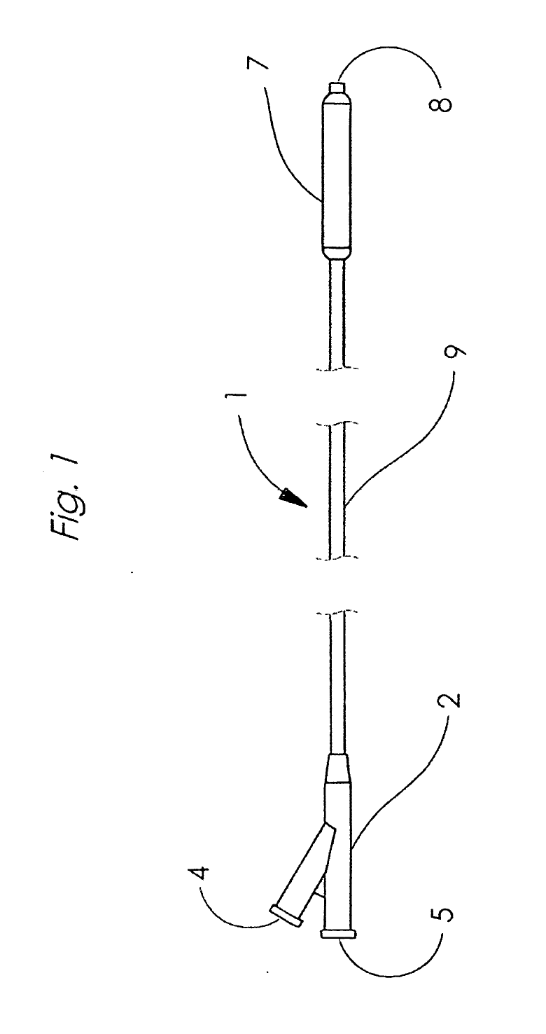

### DETAILED DESCRIPTION OF THE INVENTION

The invention relates to balloon catheter assemblies having at least one controlled failure portion located in or on the catheter shaft wall and/or the hub assembly wall and/or a hub extension piece wall. In an aspect of the invention, the catheter assembly comprises a catheter shaft having a proximal end and a distal end, an inflation lumen extending from the proximal end to the distal end, and being in fluid communication with an interior space of an inflatable balloon located on the distal end. The inflation lumen is defined by a catheter wall portion, the catheter wall portion including at least one controlled failure portion preferably located sufficiently proximate to the inflatable balloon, such that the controlled failure portion will remain inside a guide catheter or outside the patient's body when a procedure is performed. It will, of course, depend upon the particular balloon catheter device when determining the location of the controlled failure portion so that it will remain outside the patient's body or within the guide catheter during the particular procedure. For example, in the case of PTCA balloon catheters, it is generally true that the distal-most 10 cm of the catheter will, typically, extend beyond the guide catheter during a procedure. Thus, one attractive catheter assembly can comprise a catheter shaft as described above, wherein a controlled failure portion on the catheter shaft is located at least about 10 cm from the distal tip of the catheter assembly.

The catheter shaft can also comprise a guide wire receiving lumen extending for at least a portion of the length of the catheter shaft.

The invention also relates to hub assemblies for use with balloon catheters. The hub assembly comprises a hub proximal end and a hub distal end; an inflation lumen extending from the hub proximal end to the hub distal end, the inflation lumen being defined by a hub wall portion; and wherein the hub wall portion comprises at least one controlled failure portion.

Typical balloon catheter hub assemblies are made from relatively rigid plastic material such as polyvinyl chloride and polycarbonate. The proximal end of the catheter shaft is typically joined within the hub assembly, and is relatively more flexible than the hub assembly. In an aspect of the invention, the hub assembly can be releasably joined to the catheter shaft. At the distal end of the catheter shaft is located an inflatable balloon.

The invention also relates to hub extension pieces having an inflation lumen defined by a wall portion, the wall portion comprising at least one controlled failure portion. The hub extension piece can be positioned relative to a hub assembly such that the hub assembly inflation lumen is in fluid communication with the hub extension piece inflation lumen. The hub extension piece can be releasably attached to the hub assembly using any suitable means such as screwing together the pieces, or well known “quick-release” mechanisms.

The invention also relates to an assembly comprising a combination of two or more of the above-described assemblies. For example, suitable balloon catheter assemblies can comprise a catheter shaft comprising a wall having at least one controlled failure portion, as described above. Joined to the proximal end of such a catheter shaft can be a hub assembly that also includes at least one controlled failure portion. In an aspect of the invention, a controlled failure portion in the hub assembly can be designed to fail or rupture at a pressure below what would cause the controlled failure portion on the catheter shaft to fail or rupture. Of course, it would also be possible to include the hub assembly extension piece to this embodiment. The hub extension piece could be provided with a controlled failure portion that is designed to fail or rupture at a pressure below that which would cause the controlled failure portion on the hub and/or catheter shaft to fail or rupture.

This aspect of the invention may be particularly attractive in that it would allow the physician to continue with the procedure even if a first controlled failure portion fails or ruptures. For example, if an inflation pressure is reached which causes failure of a controlled failure portion on the hub wall and/or hub extension piece wall, the controlled failure portion could be plugged, covered, or otherwise sealed by any suitable means. Moreover, in the case of a hub extension piece, the hub extension piece could be quickly removed and, if desired, replaced. The procedure could then be continued without withdrawing the balloon catheter from the patient, while still having a controlled failure portion located on, for example, the catheter shaft wall, thus still providing protection against balloon rupture or failure.

When the controlled failure portion is designed to be located outside of the patient's body during the relevant procedure, it may be desirable to provide a fluid-containing device or reservoir to prevent inflation fluid from being uncontrollably discharged into the operating area. For example, a suitable fluid-containing device could comprise a polymer or plastic bag or bladder sealed about the controlled failure portion. Other suitable fluid-containing devices will be apparent to the skilled artisan.

As used herein, “controlled failure portion” includes any means located on or within the catheter shaft wall, the hub assembly wall portion, and/or the hub extension piece wall portion which will relieve pressure at a predetermined pressure and does not interfere with or interrupt inflation fluid flow through the relevant lumen. Examples of controlled failure portions include, for example, known pressure relief valves (such as spring loaded valves and check valves) as well as a frangible portion, as defined below.

The at least one “frangible portion” included in or on the catheter shaft wall, the hub wall portion of the hub assembly, or the wall of the hub extension piece is meant to mean any material which will fail or rupture (that is, relieve pressure) at a pressure below that which would cause the inflatable balloon to rupture or otherwise fail. For example, the frangible portion can be any suitable material located in or on the catheter wall, hub wall, or hub extension wall. In an aspect of the invention, “frangible portion” can include hub wall portion or hub extension wall that is thinner and/or weaker than the rest of the hub wall portion or hub extension wall, such that it will rupture, puncture, etc., at a pressure less than what would cause failure of the inflatable balloon. In the case where the frangible portion is a distinct material from the catheter wall, hub wall, or hub extension piece wall, then the frangible portion can be a disk or thin film material. By disk it is meant to include materials in shapes such as circular or oval, as well as shapes other than circular or oval, such as square shapes, rectangular shapes, or what have you. A suitable disk material can be incorporated into the catheter wall, hub wall, or hub extension wall by any suitable means. For example, a disk material can be adhered to the wall (either on the inner surface or outer surface) to cover an opening or aperture in the wall. Moreover, the disk material could be secured over the opening or aperture using a polymer film wrap about the particular wall portion. Further, the disk material could be located in the wall, for example, through any suitable molding process when the catheter shaft or hub assembly is being produced (for example, by using well-known insert molding processes). Furthermore, the frangible portion can be a polymer film material covering the opening or aperture. In an aspect of the invention, polymer film is wrapped about the wall portion to cover the opening or aperture. Suitable adhesives can be used in conjunction with the polymer film. The frangible portion will, typically, be selected to fail at a pressure well below that which would cause the inflatable balloon to rupture or fail. Failure can occur by either the disk or thin film itself rupturing, puncturing, etc., or the failure can occur at the point where the disk or thin film is joined or adhered to the wall portion.

Several advantages are realized by providing the controlled failure portion in or on the catheter wall, hub wall, or hub extension wall. For example, by providing a controlled failure mechanism in the hub, failure due to overinflation is ensured to occur outside of the body, thus avoiding all of the detrimental side effects mentioned above. Moreover, by locating a frangible portion in or on the shaft at a location that will remain within the guide catheter when in use, failure due to overinflation is ensured to occur inside the guide catheter, thus avoiding the detrimental side effects mentioned above. Furthermore, it is possible to produce a frangible portion (particularly a disk material) with far less standard deviation (i.e., higher predictability) than balloon materials, when calculating a balloon catheter's minimum rated burst strength.

Suitable frangible portion can include, as mentioned above, an embodiment wherein a portion of the hub wall or hub extension wall is thinner than the remainder of the wall portion. Moreover, as stated above, it may be desirable to incorporate a suitable disk material into or on the catheter wall, hub wall, or hub extension wall portion. In this regard, suitable disk materials comprise materials selected from the group consisting of plastics, metals, ceramics, and composite materials. Suitable metals include, for example, aluminum, copper, nickel, transition metals, iron, beryllium copper, cobalt chromium, and mixtures and alloys of these materials. In an aspect of the invention, the disk is a metal material comprising stainless steel. In a further aspect of the invention, the disk is a metal comprising temper hard aluminum of about 99.0% purity.

Further suitable disk materials include, for example, ceramics such as silicon and beryllium-based glasses, metal-containing glass films and foils, including films which are fully fired and green films (i.e., films that are not fully fired). Moreover, suitable disk materials or thin films can be organic materials such as elastomeric materials, e.g. polybutadienes, thermoplastic elastomers such as SBS, polyurethanes, and polyetheresters. Further organic materials include thermoplastics such as nylons, PEBAX, acrylic, polystyrene, blends and alloys thereof. Thermosetting materials can also be used, including, for example, thermosetting epoxy, vinyl polymers, and cyanoacrylates. Further examples of organic materials which may be suitable as a disk material include fluoropolymers, such as eFEP, FEP, PTFE, ePTFE, THV, and blends and alloys thereof. Moreover, suitable materials may include metalo-organic materials such as ceramers and polythiols. The suitable material can also be a suitable composite material. It should be understood that each material mentioned above can be mixed in some fashion with a reinforcing—or weakening—material with a separate material with aspect ratio of one or greater. Such composites might be with fibers of PTFE, polyester, polyolefin, cellulose, dacron, carbon, metal, or glass. The interface of the matrix material with the separate material may be made to be particularly weak or strong. It should be understood that a composite film might also be made by stacking more than one layer of similar or different materials, thicknesses, porosity and stress concentrations. In this regard, suitable disk materials can be porous and/or nonporous. It should be understood that each material mentioned above can be formed in some fashion such that porosity—either closed cell or open cell—can be intentionally left in the material, or essentially removed.

Finally, suitable disk material or thin films can be manufactured and prescored for stress concentration in the disk or thin film material. In this regard, it should be understood that each material can be intentionally scored or otherwise weakened by leaving a stress concentrator on the disk or film itself.

Disk or thin film dimensions will, of course, depend on any number of variables including, for example, the material used and the desired failure pressure. One particularly attractive disk material could be temper hard aluminum of about 99.0% purity having an about 0.5 inch diameter and a nominal thickness of 0.025 mm.

The inflatable balloon will typically comprise a suitable polymer material. Suitable polymer materials include, for example, materials comprising a material selected from the group consisting of polytetrafluoroethylene, polyolefin copolymer, polyester, polyethylene pterothalate, polyethylene, polyether block amide, polyamide, polyimide, latex, urethane, and combinations or blends thereof. In as aspect of the invention, the balloon material comprises expanded polytetrafluoroethylene. In a further aspect of the invention, the balloon material comprises expanded polytetrafluoroethylene and elastomer, such as disclosed in U.S. Pat. No. 6,120,477.

The invention will find use in any number of catheter applications, wherein rupturing an inflatable balloon is a possibility. For example, catheters which would particularly benefit from the controlled failure mechanism of the present invention include, for example, dilatation catheters such as PTA, PTCA, and even balloon catheters used for cerebral applications. Such catheters would include over-the-wire catheters, rapid exchange catheters, and the more recently introduced convertible-type catheters, which can be used in either the over-the-wire or rapid exchange mode. Furthermore, balloon catheters used to deliver stents and/or stent grafts may also benefit from the present invention. Further catheter devices include occlusion balloon catheters and balloon catheters used for thrombus removal.

Referring now to the figures, particularly preferred embodiments will be described in detail.

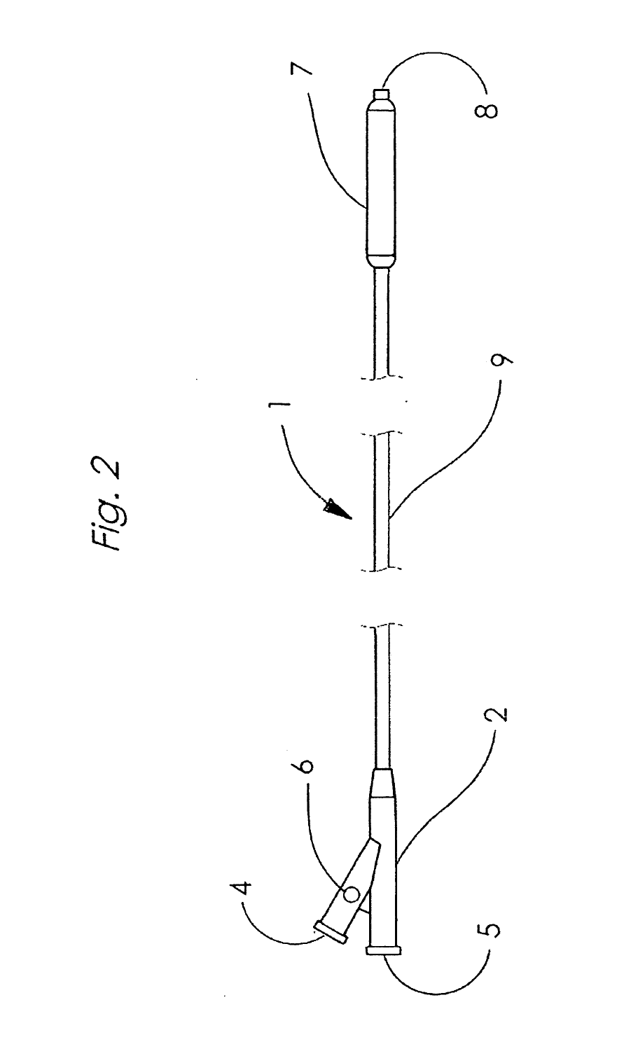

FIG. 2 shows an over-the-wire type balloon dilatation catheter that could be used in either PTA, PTCA, or cerebral applications. Catheter assembly 1 includes Y-shaped hub assembly 2 which includes guide wire port 5 and inflation fluid port 4. Hub assembly 2 comprises a proximal end and a distal end and controlled failure portion 6. The hub assembly is joined to the catheter shaft 9 which has located on the distal end thereof an inflatable balloon 7, wherein a guide wire lumen extends through the balloon and terminates at a guide wire exit port at catheter distal tip 8.

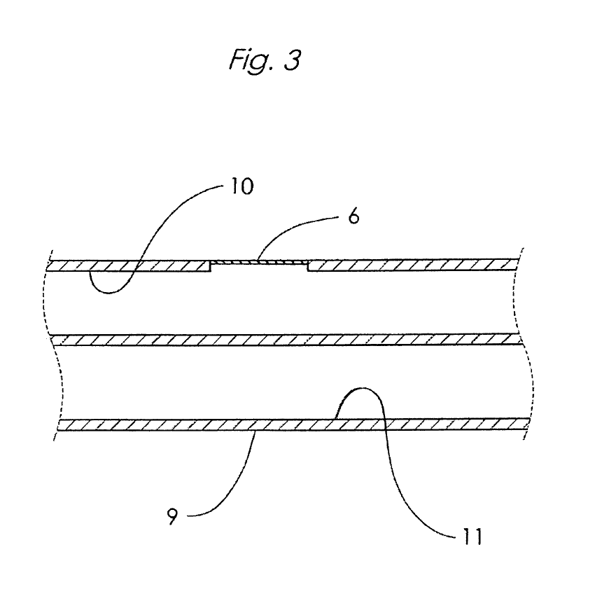

Shown in cross section in FIG. 3 is a portion of catheter shaft 9 comprising guide wire receiving lumen 11 and inflation lumen 10. Inflation lumen 10 is defined by a wall portion that includes frangible portion 6. Frangible portion 6 can be any suitable disk material or thin film material, as discussed above, located in the wall, on the outside surface of the wall, or on the inside surface of the wall.

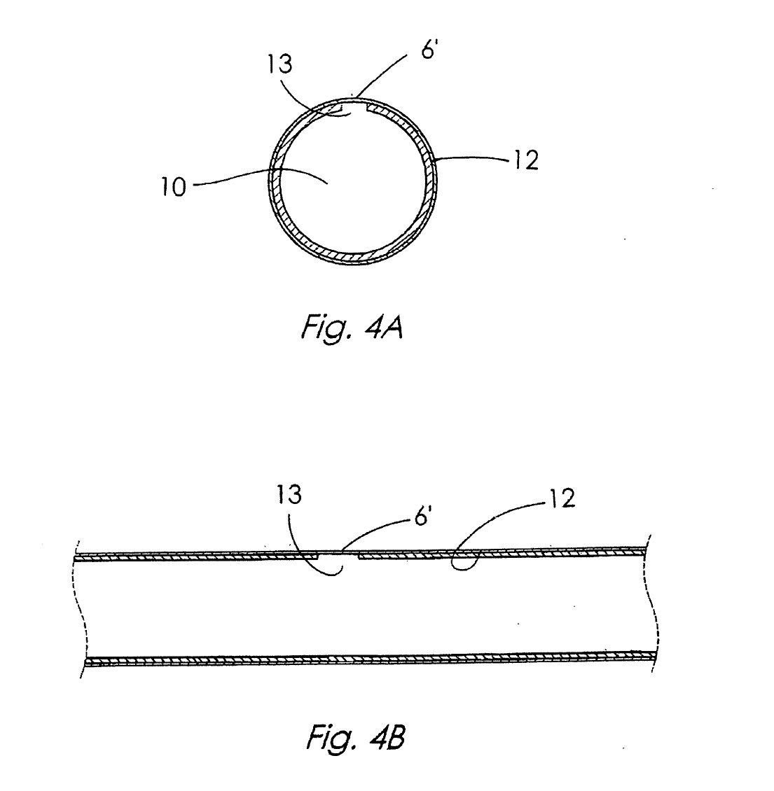

In an aspect of the invention, frangible portion can be a tubular member located on the outside of the wall portion that defines inflation lumen 10. For example, as shown in cross section in FIG. 4A, tubular member 6′ is shown in contact with the outer surface of wall portion 12 and completely covering the opening or aperture 13 provided in wall portion 12. Tubular member 6′ can be selectively adhered to portions of wall portion 12 to insure a sufficient bond between the materials to prevent undesired failure. Tubular member 6′ can be configured to provide structural support to the wall portion if desired and can extend any desired length of the catheter shaft. In as aspect of the invention tubular member 6′ can extend essentially from the proximal end of inflation lumen 10 to the distal end. Moreover, tubular member 6′ can be engineered to have selected degrees of flexibility from a proximal point to a distal point thereon. Tubular member 6′ can be any frangible material discussed above, e.g., metal, polymer, etc.

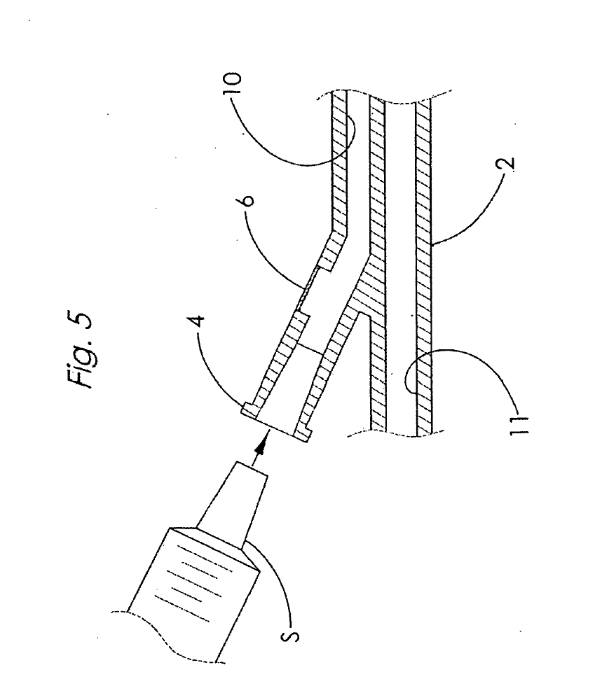

Shown in cross section in FIG. 5 is hub assembly 2 comprising guide wire receiving lumen 11 and inflation lumen 10. As can be seen, inflation lumen 10 is defined by a wall portion, wherein the wall portion includes controlled failure portion 6. Also shown at the proximal end of the hub assembly is inflation port 4 which is designed to receive a suitable inflation device, such as syringe S, or a hub extension piece (not shown).

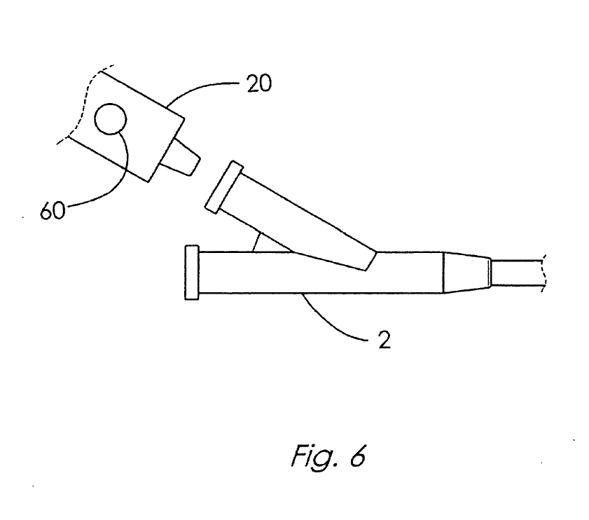

Turning to FIG. 6, there is shown hub assembly 2 and hub extension piece 20. Hub extension piece 20 defines an inflation lumen extending from its proximal end to the distal end, wherein controlled failure portion 60 is provided in or on the wall portion of hub extension piece 20.

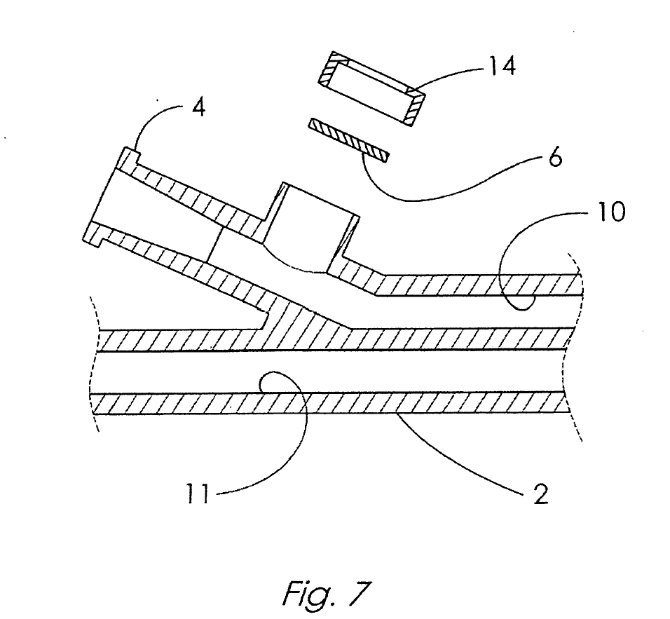

FIG. 7 shows a further aspect of the invention wherein the controlled failure portion is located somewhat offset or protruding from the wall portion. As can be seen, controlled failure portion comprises disk material 6 which is locatable on a portion of protruding hub wall portion. In an aspect of the invention, the disk material can be secured to the hub wall by providing a securable cover 14, such as a snap-on cover, twist-on cover, or other variation thereof. It will be appreciated that such an embodiment will provide for easy and quick changing of the disk material, which is simply a “drop-in” component. Moreover, this embodiment can provide a mode for easily securing a suitable fluid-containing device to the catheter, hub assembly, or hub extension piece. For example, the opening to a suitable bag or bladder could be tightly and removably sealed to the protruding portion of the wall.

As discussed above, controlled failure portion 6 is designed to rupture, puncture, or otherwise fail at a pressure below the rupture pressure of the inflatable balloon.

In addition to providing for a safe and controlled failure mode mechanism for balloon catheters, the present invention also provides for producing catheters with higher minimum burst strengths than identical catheters without the controlled failure portion of the present invention. This can be a particular benefit wherein the balloon material of choice results in a relatively high standard deviation, as discussed below. Balloon minimum burst strength can be determined as follows:

Balloon Minimum Burst Strength: Determine the rated burst pressure for each balloon size (i.e., each balloon diameter and length combination). This test should be conducted on complete catheters or subassemblies in which the balloon is mounted on the catheter shaft. Any loss of pressure, whether due to failure of the balloon, shaft or proximal or distal seals, should be considered a failure in this test. The pressure at which the failure occurred and the failure mode should be recorded. The rated burst pressure is based on the results of the balloon burst testing, which shows statistically with at least 95% confidence that 99.9% of the balloons will not burst at or below the minimum burst pressure. Below is one formula for determining rated burst pressure.

Using a one-sided tolerance limit for a normal distribution:

Let P=0.999 (99.9%)
C=0.95 (95% confidence)
N=number of balloons tested
   K=factor for a one-sided tolerance limit for a normal distribution (K is found in statistical tables and is dependent on P, C and N above)
   X=mean balloon burst strength
   SD=standard deviation
      X−K(SD)=minimum burst strength
      The rated burst pressure is some arbitrary pressure below the minimum burst pressure. As a safety factor, manufacturers typically use a rated burst pressure which is at least one standard deviation lower than the minimum burst strength.

From the above, it can be seen that the minimum burst strength is highly dependent upon the standard deviation of the particular materials used. That is, a higher standard deviation will result in a lower minimum burst strength. Thus, it should be apparent to the skilled artisan that by carefully selecting the proper controlled failure mechanism, and particularly the frangible portion, and more particularly, a suitable disk material that will fail at a pressure below the balloon burst pressure, but with far higher predictability (i.e., less standard deviation), it will be possible to produce balloon catheter devices having a higher rated minimum burst strength. In this regard, thin stainless steel or aluminum disk materials may be particularly attractive for PTCA balloon catheter devices. Such balloon catheter devices are typically routinely inflated to pressures of about 18 atmospheres.

1-16. (canceled)

17. Catheter assembly comprising:
a catheter shaft having a proximal end and a distal end, an inflation lumen extending from the proximal end to the distal end, and being in fluid communication with an interior space of an inflatable balloon located on the distal end, the inflation lumen being defined by a wall portion, the wall portion including at least one frangible portion located sufficiently proximate to the inflatable balloon such that the at least one frangible portion will remain inside a guide catheter when a procedure is performed in a patient's vasculature.

18. The catheter assembly of claim 17, wherein the catheter shaft further comprises a guide wire receiving lumen extending from the distal end to a point proximate to the inflatable member.

19. The catheter assembly of claim 17, wherein the at least one frangible portion comprises a disk material comprising a material selected from the group consisting of plastics, metals, ceramics and composite materials.

20. The catheter assembly of claim 19, wherein the metals are selected from the group consisting of aluminum, copper, nickel, transition metals, iron, beryllium copper, cobalt chromium, and mixtures and alloys thereof.

21. The catheter of claim 20, wherein the metal comprises stainless steel.

22. The catheter assembly of claim 17, wherein the inflatable balloon comprises a polymer material.

23. The catheter assembly of claim 22, wherein the polymer material comprises a material selected from the group consisting of polytetrafluoroethylene, polyolefin copolymer, polyester, polyethylene terephthalate, polyethylene, polyether block amide, polyamide, polyimide, latex, urethane, and combinations or blends thereof.

24. The catheter assembly of claim 23, wherein the polytetrafluoroethylene comprises expanded polytetrafluoroethylene.

25. The catheter of claim 17, wherein a stent is mounted on the inflatable balloon.

26. The catheter of claim 17, wherein the inflatable balloon is a dilatation balloon.

27. The catheter of claim 17, wherein the inflatable balloon is an occlusion balloon.

28. The catheter of claim 17, wherein the catheter shaft further comprises a guide wire receiving lumen extending from the distal end to a point proximal of the hub assembly.

29. The catheter of claim 18, wherein the catheter shaft includes a guide wire receiving lumen extending from the distal end to the proximal end and being in fluid communication with the hub assembly guide wire receiving lumen.

30-43. (canceled)

44. The catheter assembly of claim 17, wherein the catheter minimum rated burst strength is increased by the at least one frangible portion relative to the same catheter assembly without the at least one frangible portion.

45. (canceled)
